(19) United States
(12) Patent Application Publication
(10) Pub. No.: US 2020/0173561 A1
(43) Pub. Date: Jun. 4, 2020

(54) ELASTIC SEALING DEVICE, FIXING STRUCTURE OF ELASTIC SEALING DEVICE, AND ENDOSCOPE

(71) Applicant: OLYMPUS CORPORATION, Tokyo (JP)

(72) Inventors: Yosuke NAKAO, Tokyo (JP); Tatsuyuki OGAWA, Tokyo (JP); Takanori USHIJIMA, Tokyo (JP); Kaoru TSURUOKA, Kawasaki-shi (JP)

(73) Assignee: OLYMPUS CORPORATION, Tokyo (JP)

(21) Appl. No.: 16/715,613

(22) Filed: Dec. 16, 2019

Related U.S. Application Data
(63) Continuation of application No. PCT/JP2018/023966, filed on Jun. 25, 2018.

(30) Foreign Application Priority Data
Jun. 30, 2017 (JP) 2017-129044

(51) Int. Cl. F16J 15/3268; A61B 1/00

(52) U.S. Cl. CPC F16J 15/3268; A61B 1/122; A61B 1/00121; A61B 1/00071

(57) ABSTRACT

An elastic sealing device includes a watertight holding section that is provided between adjacent frame bodies and holds watertightness and a packing section that is projectingly provided with respect to the watertight holding section and that is arranged in a gap between the adjacent frame bodies and forms a continuous outer surface.

### CROSS REFERENCE TO RELATED APPLICATION

This application is a continuation application of PCT/JP2018/023966 filed on Jun. 25, 2018 and claims benefit of Japanese Application No. 2017-129044 filed in Japan on Jun. 30, 2017, the entire contents of which are incorporated herein by this reference.

### BACKGROUND OF INVENTION

### 1. Field of the Invention

The present invention relates to an elastic sealing device including a watertight holding section that holds watertightness between adjacent frame bodies and a packing section that fills a gap between frame bodies, a fixing structure of the elastic sealing device, and an endoscope.

### 2. Description of the Related Art

An endoscope has been used in a medical field, an industrial field, and the like. In the endoscope used in the medical field, an elongated insertion section is inserted into a body. Therefore, the endoscope that has been once used for an inspection, a treatment, or the like is cleaned and then a sterilization treatment is implemented. Therefore, the endoscope has a watertight structure for preventing water, etc. from penetrating into the inside.

A composition of the endoscope such as an endoscope operation section or an endoscope connector is configured by assembling a tubular or cylindrical frame body. For example, the operation section is configured by assembling a plurality of frame bodies. Further, as shown in FIG. 1A, a circumferential groove 5 to which an O-shaped ring 4 is attached is formed in one end of an engaging section 3 in which a first frame body 1 and a second frame body 2 respectively are arranged.

As shown in FIG. 1B, in a state in which the first frame body 1 and the second frame body 2 are assembled, an engaging section 3 between the first frame body 1 and the second frame body 2 is brought into a state in which watertightness is held by the O-shaped ring 4. In the state in which the watertightness is held, an outer surface of the first frame body 1 and an outer surface of the second frame body 2 are preferably formed as a continuous outer surface without steps in order to facilitate cleaning of the endoscope. In other words, a distal end surface 1f of the first frame body 1 and a proximal end surface 2r of the second frame body 2 are preferably arranged without gaps.

When the endoscope is sterilized using an autoclave, the endoscope is exposed to a high-temperature and high-pressure atmosphere and the first frame body 1 and the second frame body 2 thermally expand in a longitudinal axis direction. As described above, when the distal end surface if of the first frame body 1 and the proximal end surface 2r of the second frame body 2 are arranged without gaps, a load is applied to an abutment surface between the distal end surface 1f and the proximal end surface 2r. Further, the autoclave sterilization in the endoscope is repeatedly performed, which may cause a phenomenon such that either the distal end surface if of the first frame body 1 or the proximal end surface 2r of the second frame body 2 is cracked, broken, or the like depending on materials.

A gap for preventing the distal end surface if and the proximal end surface 2r from abutting on each other due to a thermal expansion of the first frame body 1 and the second frame body 2 during sterilization by steam is previously provided between the distal end surface if and the proximal end surface 2r shown in FIG. 1B to thereby solve an occurrence of the phenomenon.

Note that Japanese Patent Application Laid-Open Publication No. 2005-230436 discloses a battery unit in which performance of a battery is not deteriorated in a high-temperature state of an external environment but the performance of the battery is improved in a high-temperature state due to a pressurized steam of the external environment.

### SUMMARY OF THE INVENTION

A fixing structure of an elastic sealing device according to one aspect of the present invention includes an elastic sealing device including a watertight holding section that is provided between adjacent frame bodies and holds watertightness and a packing section that is projectingly provided with respect to the watertight holding section and that is arranged in a gap between the adjacent frame bodies and forms at least a part of an outer surface, a first frame body including a first frame outer peripheral surface that is exposed to an outside and is an outer surface and a first frame engaging surface in which the elastic sealing device is arranged, and a second frame body including a second frame outer peripheral surface that is exposed to the outside and is an outer surface and a second frame engaging surface that is arranged facing the first frame engaging surface, and in which a gap for arranging the elastic sealing device is formed between the first frame engaging surface and the second frame engaging surface by being assembled into the first frame body.

An elastic sealing device according to one aspect of the present invention includes a watertight holding section that is provided between adjacent frame bodies and holds watertightness, and a packing section that is projectingly provided with respect to the watertight holding section and that is arranged in a gap between the adjacent frame bodies and forms at least a part of an outer surface, wherein the watertight holding section includes, in a circumferential direction, a plurality of convex sections that project in a direction opposite to the packing section from the watertight holding section.

Further, an elastic sealing device according to another aspect of the present invention includes a watertight holding section that is provided between adjacent frame bodies and holds watertightness, and a packing section that is projectingly provided with respect to the watertight holding section and that is arranged in a gap between the adjacent frame bodies and forms at least a part of an outer surface, wherein the packing section is a circumferential projection that projects from the watertight holding section in one direction and is provided along an outer peripheral surface or an inner peripheral surface of the watertight holding section, and a central line of the circumferential projection and a central line of the watertight holding section do not match each other.

An endoscope according to one aspect of the present invention is an endoscope that includes a fixing structure of an elastic member including an elastic sealing device including a watertight holding section that is provided between adjacent frame bodies and holds watertightness and a packing section that is projectingly provided with respect to the watertight holding section and that is arranged in a gap between the adjacent frame bodies and forms at least a part of an outer surface, a first frame body including a first frame outer peripheral surface that is exposed to an outside and is an outer surface and a first frame engaging surface in which the elastic sealing device is arranged, and a second frame body including a second frame outer peripheral surface that is exposed to the outside and is an outer surface and a second frame engaging surface that is arranged facing the first frame engaging surface, and in which a gap for arranging the elastic sealing device is formed between the first frame engaging surface and the second frame engaging surface by being assembled into the first frame body, in an endoscope composition such as an endoscope operation section or an endoscope connector.

An endoscope according to another aspect of the present invention is an endoscope in which an elastic sealing device that includes a watertight holding section that is provided between adjacent frame bodies and holds watertightness, and a packing section that is projectingly provided with respect to the watertight holding section and that is arranged in a gap between the adjacent frame bodies and forms at least a part of an outer surface and in which the watertight holding section includes, in a circumferential direction, a plurality of convex sections that project in a direction opposite to the packing section from the watertight holding section, is provided in at least an endoscope operation section or an endoscope connector.

An endoscope according to another aspect of the present invention is an endoscope in which an elastic sealing device that includes a watertight holding section that is provided between adjacent frame bodies and holds watertightness, and a packing section that is projectingly provided with respect to the watertight holding section and that is arranged in a gap between the adjacent frame bodies and forms at least a part of an outer surface and in which the packing section is a circumferential projection that projects from the watertight holding section in one direction and is provided along an outer peripheral surface or an inner peripheral surface of the watertight holding section, and a central line of the circumferential projection and a central line of the watertight holding section do not match each other, is provided in at least an endoscope operation section or an endoscope connector.

### BRIEF DESCRIPTION OF THE DRAWINGS

FIG. 1A is a diagram describing a first frame body and a second frame body before assembling;

FIG. 1B is a diagram describing a state in which the first frame body and the second frame body are assembled;

FIG. 2A is a front view of an elastic sealing device;

FIG. 2B is a cross-sectional view along a line Y2B-Y2B shown in FIG. 2A and is a diagram describing a configuration in which a packing main body and a joining section configuring a packing section are made to have the same thickness and a thin wall section is thinner than the packing section;

FIG. 3A is a diagram describing the first frame body and the second frame body configuring an endoscope operation section;

FIG. 3B is a diagram describing a positional relationship between a first frame engaging surface of the first frame body and the elastic sealing device;

FIG. 3C is a diagram describing a gap configured when arranging an engaging convex section of the second frame body in an engaging concave section of the second frame body;

FIG. 4A is a diagram describing a state in which the elastic sealing device shown in FIGS. 2A and 2B is attached to a first frame engaging surface of the first frame body;

FIG. 4B is a diagram describing a state in which the second frame body is engaged with the first frame body to which the elastic sealing device is attached;

FIG. 4C is a diagram describing a relationship between a thickness of the packing main body and a thickness of the joining section configuring the packing section in a state in which the elastic sealing device is stably pressed and held between the first frame body and the second frame body in the state in which the first frame body and the second frame body are assembled;

FIG. 4D is a diagram describing a relationship among the first frame body, the second frame body, and the elastic sealing device in a high-temperature and high-pressure atmosphere during autoclave sterilization;

FIG. 4E is a diagram describing the second frame body in which a second engaging flat surface is an inclined flat surface;

FIG. 4F is a diagram describing an action of the second engaging flat surface that is the inclined flat surface;

FIG. 4G is a diagram describing a relationship between a compression rate of the packing main body and a compression rate of the joining section configuring the packing section in a state in which the elastic sealing device is stably pressed and held between the first frame body and the second frame body in the state in which the first frame body and the second frame body are assembled;

FIG. 5A is a diagram describing the elastic sealing device in which a thickness is thinly set in the order corresponding to the packing main body, the joining section, and the thin wall section configuring the packing section;

FIG. 5B describes a relationship between the first frame body and the second frame body in a state in which the elastic sealing device shown in FIG. 5A is attached to the first frame engaging surface of the first frame body;

FIG. 5C is a diagram describing a state in which the second engaging flat surface of the second frame body is caused to be pressed and arranged on the other surface of the packing main body of the elastic sealing device;

FIG. 6 is a diagram describing the elastic sealing device in which a concave section is provided in the packing section;

FIG. 7A is a diagram describing a configuration example of the packing section that prevents the packing main body from protruding to the outside of the first frame body and the second frame body;

FIG. 7B is a diagram describing the elastic sealing device in the state in which the first frame body and the second frame body are assembled;

FIG. 8A is a diagram describing another configuration of the packing section that prevents the packing main body from protruding to the outside of the first frame body and the second frame body;

FIG. 8B is a diagram describing the elastic sealing device in the state in which the first frame body and the second frame body are assembled;

FIG. 9A is a diagram describing the other configuration of the packing section that prevents the packing main body from protruding to the outside of the first frame body and the second frame body;

FIG. 9B is a diagram describing the elastic sealing device in the state in which the first frame body and the second frame body are assembled;

FIG. 10A is a front view of the elastic sealing device according to the other configuration of the elastic sealing device;

FIG. 10B is a cross-sectional view along a line Y6B-Y6B of arrows shown in FIG. 10A;

FIG. 10C is a back view of the elastic sealing device;

FIG. 11A is a diagram describing a relationship among the elastic sealing device shown in FIGS. 10A to 10C, the first frame body, and the second frame body;

FIG. 11B is a diagram describing a state in which the second frame body is engaged with the first frame body to which the elastic sealing device is attached;

FIG. 12A is a diagram describing the elastic sealing device in which an escape section is provided; and

FIG. 12B is a diagram describing the elastic sealing device in which a positioning concave section or an attachment section in consideration of a working property is provided.

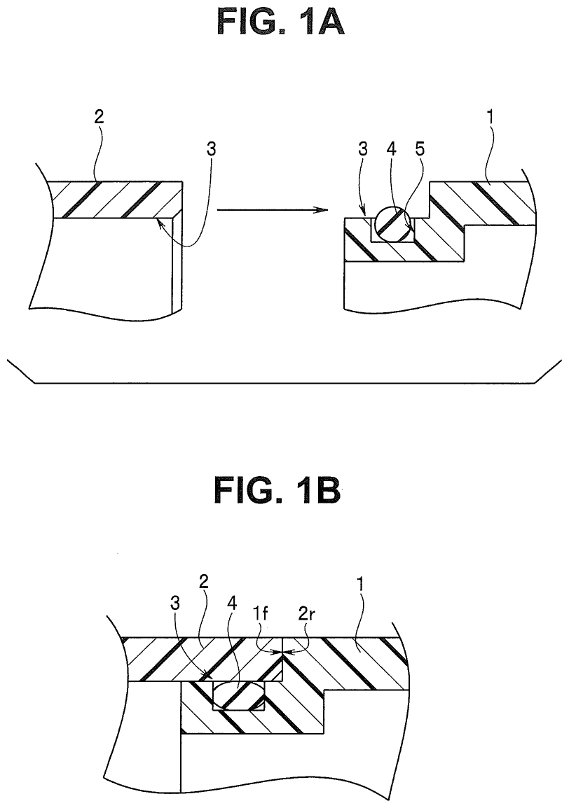

### DETAILED DESCRIPTION OF THE PREFERRED EMBODIMENTS

Hereinafter, an embodiment of the present invention will be described with reference to the accompanying drawings.

In each drawing used in the following description, a scale of each component may be different in order to make each component recognizable in the drawings. In other words, the present invention is not limited only to the number of the components, shapes of the components, ratios among sizes of the components, and relative positional relationships among the components described in these drawings.

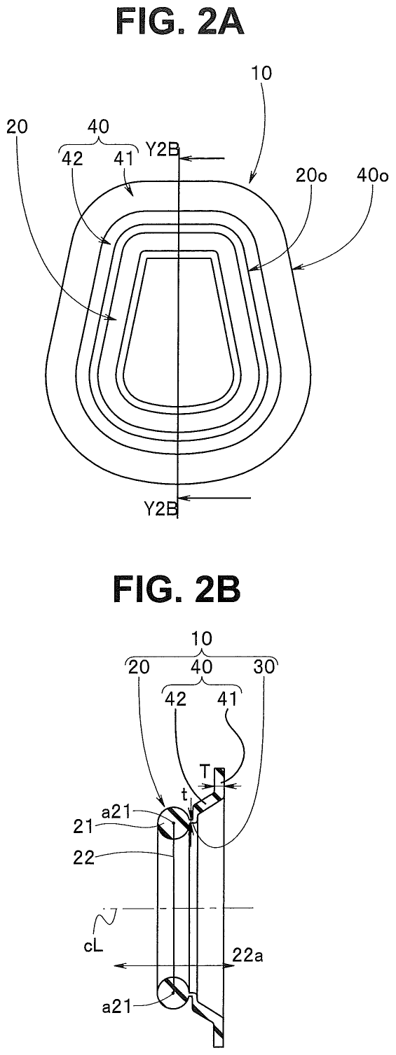

An elastic sealing device 10 will be described with reference to FIGS. 2A and 2B.

The elastic sealing device 10 is formed by using a raw material that is excellent in heat resistance and has a predetermined rubber hardness. The elastic sealing device 10 includes a watertight holding section 20, a thin wall section 30, and a packing section 40 shown in FIGS. 2A and 2B and is formed in a substantially ring shape.

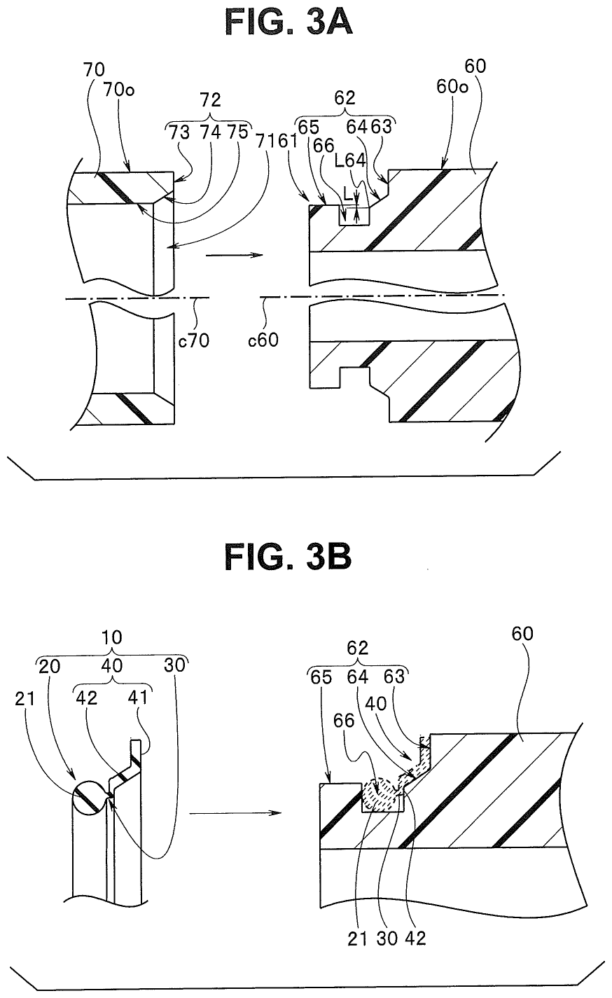

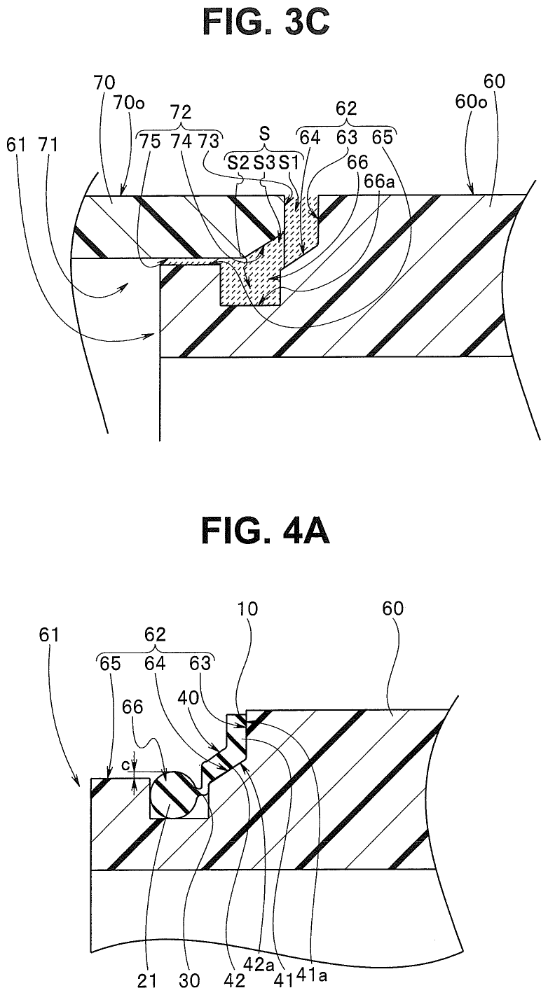

The elastic sealing device 10 is attached to an engaging convex section 61 of a first frame body 60 shown in FIG. 3A.

As shown in FIG. 2A, an outside surface 40o of the packing section 40 is formed more largely than an outside surface 20o of the watertight holding section 20. As shown in FIG. 2B, the watertight holding section 20 is an O-shaped ring shape section 21 a cross-sectional shape of which is a substantially circular form. A cross-sectional shape of the packing section 40 is of a crooked shape and the packing section 40 is provided on one side 22a of the watertight holding section 20 along a central line cL orthogonal to a middle point of a central axis segment 22 connecting central axes a21 of the O-shaped ring shape section 21.

As shown in FIGS. 2A and 2B, the packing section 40 includes a packing main body 41 that is a first portion and a joining section 42 that is a second portion. The joining section 42 is provided so as to be positioned between the watertight holding section 20 and the packing main body 41. Further, the thin wall section 30 is provided between the watertight holding section 20 and the joining section 42.

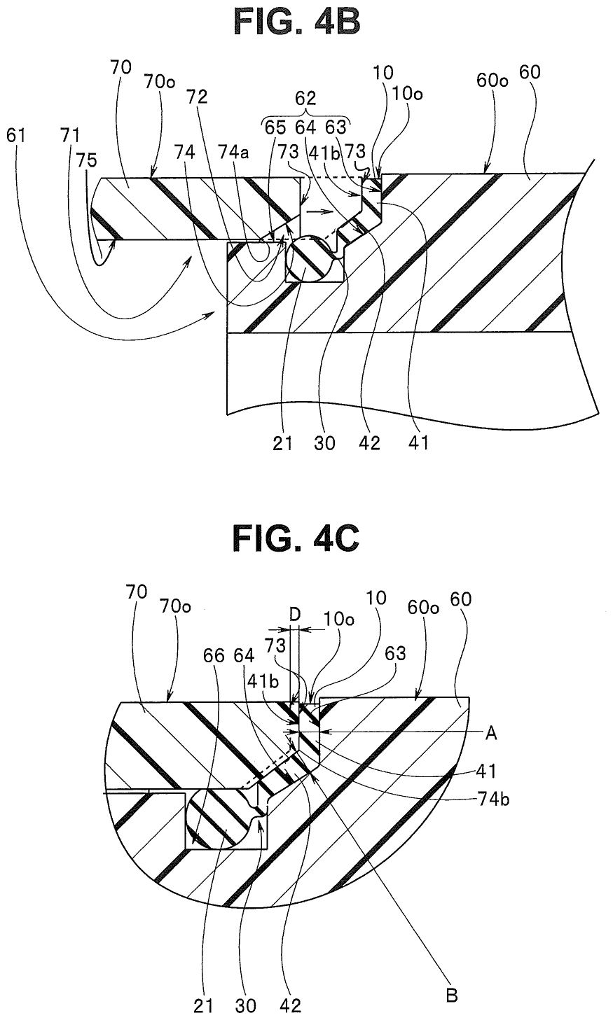

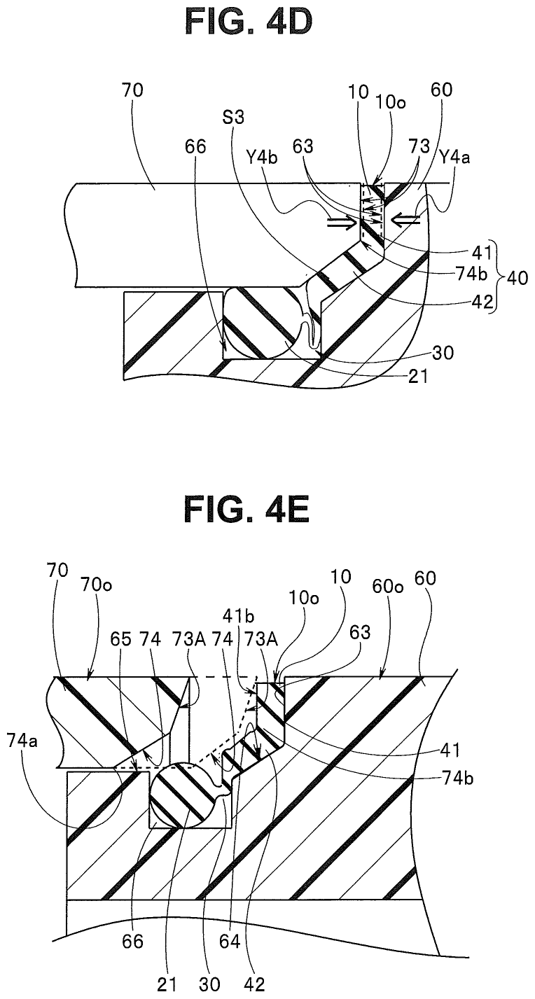

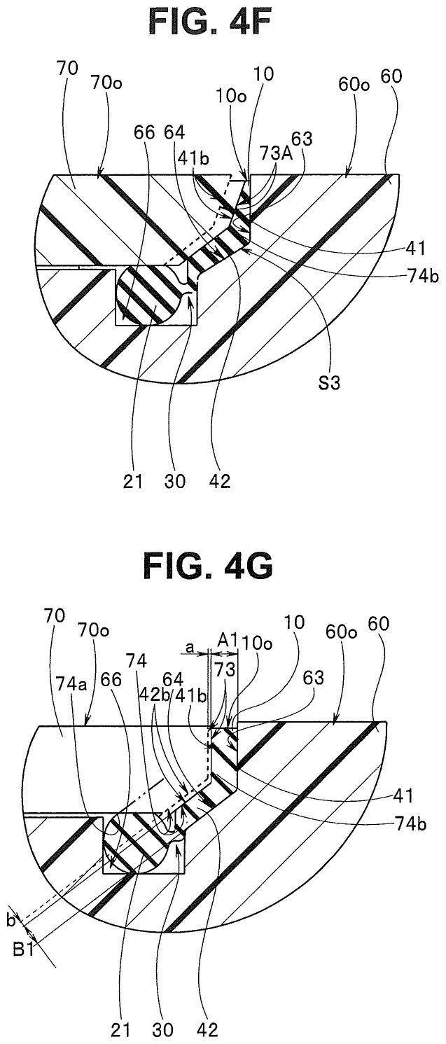

Note that a projection position from the O-shaped ring shape section 21 of the thin wall section 30 is arranged in a circumferential groove 66 as shown in FIG. 4A to be described later.

As shown in FIG. 2B, the packing main body 41 has a flat surface orthogonal to the central line cL. On the other hand, the joining section 42 has an inclined surface inclined to the central line cL. A thickness t of the thin wall section 30 coupling the watertight holding section 20 and the joining section 42 is set to be smaller than a thickness T of the packing section 40. According to the present embodiment, the packing main body 41 and the joining section 42 have the same thickness T.

The first frame body 60 and a second frame body 70 configuring an endoscope operation section will be described with reference to FIGS. 3A and 3B.

Reference numerals 60 and 70 shown in FIG. 3A represent the first frame body 60 and the second frame body 70 configuring the endoscope operation section and the first frame body 60 and the second frame body 70 are provided adjacent to each other. The engaging convex section 61 is provided on the first frame body 60 and an engaging concave section 71 with which the engaging convex section 61 is engaged is provided on the second frame body 70.

A reference numeral 60o represents an external surface that is a first frame outer peripheral surface and is exposed to the outside. A reference numeral 70o represents an external surface that is a second frame outer peripheral surface and is exposed to the outside. A reference numeral c60 represents a first longitudinal axis of the first frame body 60. A reference numeral c70 represents a second longitudinal axis of the second frame body 70.

The engaging convex section 61 includes a first frame engaging surface 62 for arranging the elastic sealing device 10. The first frame engaging surface 62 includes a first engaging flat surface 63, a first engaging inclined surface 64, and an engaging side outer peripheral surface 65.

The first engaging flat surface 63 that is an engaging surface is a flat surface that intersects with the first longitudinal axis c60 and the first engaging flat surface 63 according to the present embodiment is a vertical flat surface orthogonal to the first longitudinal axis c60. The first engaging inclined surface 64 intersects with the first longitudinal axis c60 and is an inclined surface that intersects with the first engaging flat surface 63. The engaging side outer peripheral surface 65 is an outer peripheral surface at the center of the first longitudinal axis c60. The circumferential groove 66 is formed in the engaging side outer peripheral surface 65. The circumferential groove 66 is formed in a predetermined width and depth.

A formation position of a crossing ridge line L64 between the circumferential groove 66 and the first engaging inclined surface 64 is positioned to the first longitudinal axis c60 side by a predetermined distance L from the position of the engaging side outer peripheral surface 65.

The elastic sealing device 10 is arranged on the first frame engaging surface 62 described above. Specifically, as shown in FIG. 3B, the packing section 40 is arranged on the first engaging flat surface 63 and the first engaging inclined surface 64. Further, the O-shaped ring shape section 21, the thin wall section 30, and a thin wall side end section of the joining section 42 that is a part of the packing section 40 are arranged in the circumferential groove 66.

As shown in FIG. 3A, a second frame engaging surface 72 is provided in the second frame body 70. The second frame engaging surface 72 is arranged facing the first frame engaging surface 62 and includes a second engaging flat surface 73, a second engaging inclined surface 74, and an engaging side inner peripheral surface 75.

The second engaging flat surface 73 that is an engaging surface is a distal end surface of the second frame body 70. Further, the second engaging flat surface 73 is a flat surface that intersects with the second longitudinal axis c70 and is a vertical flat surface orthogonal to the longitudinal axis c70. The second engaging inclined surface 74 is an inclined surface that intersects with the second longitudinal axis c70 and intersects with the second engaging flat surface 73. The engaging side inner peripheral surface 75 is an inner peripheral surface at the center of the second longitudinal axis c70.

As shown in FIG. 3C, in an engaging state in which the engaging convex section 61 of the first frame body 60 is arranged in the engaging concave section 71 of the second frame body 70, a gap S is configured between the first frame body 60 and the second frame body 70 adjacent to each other. The elastic sealing device 10 is arranged in the gap S.

Note that the gap S is configured by a first gap S1 in which the first engaging flat surface 63 faces the second engaging flat surface 73, a second gap S2 in which the engaging side outer peripheral surface 65 and an inner peripheral surface 66a in the circumferential groove 66 face the engaging side inner peripheral surface 75, and a third gap S3 in which the first engaging inclined surface 64 faces the second engaging inclined surface 74.

Here, assembling of the endoscope operation section will be described.

An operator prepares the first frame body 60, the second frame body 70, and the elastic sealing device 10. As shown in FIG. 4, the operator first arranges the elastic sealing device 10 on the first frame engaging surface 62 of the engaging convex section 61. At this time, the operator causes the O-shaped ring shape section 21 of the elastic sealing device 10 to be arranged in the circumferential groove 66. At the same time, the operator causes the packing section 40 to be arranged on the first engaging flat surface 63 and the first engaging inclined surface 64 of the first frame engaging surface 62.

Here, the operator performs fine-tuning and causes the thin wall section 30 and the thin wall side end section of the joining section 42 that is a part of the packing section 40 to be housed and arranged in the circumferential groove 66. Then, the operator causes one surface 41a of the packing main body 41 to abut on the first engaging flat surface 63. Further, the operator causes one surface 42a of the joining section 42 to abut on the first engaging inclined surface 64. At this time, a top of the O-shaped ring shape section 21 is positioned in a state of being projected, by c, from the engaging side outer peripheral surface 65.

Next, as shown in FIG. 4B, the operator arranges the engaging convex section 61 of the second frame body 60 in the engaging concave section 71 of the second frame body 70. The process permits the first frame body 60 and the second frame body 70 to be brought into an engaging state. Here, the operator causes the second engaging flat surface 73 to move to the first engaging flat surface 63 as shown in an arrow.

Then, a first ridge line 74a in which the second engaging inclined surface 74 intersects with the engaging side inner peripheral surface 75 abuts on the O-shaped ring shape section 21 projected from the engaging side outer peripheral surface 65. At this time, the second frame body 70 includes the second engaging inclined surface 74, and thereby the second frame body 70 is further moved to the first engaging flat surface 63 while easily squeezing the O-shaped ring shape section 21. Then, as shown in a broken line, the second engaging flat surface 73 abuts on the other surface 41b of the packing main body 41 of the elastic sealing device 10.

As described above, the O-shaped ring shape section 21 is squeezed and thereby the O-shaped ring shape section 21 can be attached closely to the inner peripheral surface 66a in the circumferential groove 66 provided in the engaging side outer peripheral surface 65 and the engaging side inner peripheral surface 75, and the first frame body 60 and the second frame body 70 in the engaging state can be brought into a watertight holding state.

Here, as shown in FIG. 4C, the operator moves, by a distance D, the second engaging flat surface 73 to the first engaging flat surface 63. Then, the second engaging flat surface 73 presses the other surface 41b of the packing main body 41, and thereby a thickness T of the packing main body 41 is compressed and deformed to a size A. On the other hand, the packing main body 41 is extended and deformed along with the compression. Specifically, the packing main body 41 is extended to the O-shaped ring shape section 21 side from a second ridge line 74b in which the second engaging flat surface 73 intersects with the second engaging inclined surface 74.

The packing main body 41 is extended and thereby the thickness T of a part of the packing main body 41 and a part of the joining section 42 arranged in the third gap S3 is expanded to a size B. Further, the part of the packing main body 41 and the part of the joining section 42 are deformed so as to be attached closely to the second engaging inclined surface 74 of the second frame body 70. Here, a relationship between the size A and the size B satisfies an expression A<B.

At this time, a close contact state of eliminating the gap is brought between the second engaging flat surface 73 and the other surface 41b of the packing main body 41 and between the one surface 41a of the packing main body 41 and the first engaging flat surface 63. At this time, a surface on the outer side of the packing main body 41 arranged between the first frame body 60 and the second frame body 70 configures a seal member outer peripheral surface 10o.

According to the configuration, in an assembled state between the first frame body 60 and the second frame body 70, the packing section 40 of the elastic sealing device 10 is brought into a state of biting into the second ridge line 74b and is stably pressed and held between the first frame body 60 and the second frame body 70.

Thereby, the joining section 42 can be firmly fixed between the first frame body 60 and the second frame body 70. Further, by a load at the time of being charged in an autoclave or the like, the packing main body 41 can be prevented from protruding to an outer side of the first frame body 60 and an outer side of the second frame body 70.

In this state, the operator integrally fixes the first frame body 60 and the second frame body 70, for example, by using a setscrew, an adhesive agent, or the like that is not shown to thereby complete the assembling.

Note that, in the assembled state, the seal member outer peripheral surface 10o of the elastic sealing device 10, a first frame outer peripheral surface 60o, and a second frame outer peripheral surface 70o may be configured as a continuous outer surface without steps, and further, are made concave in a range in which the surfaces can be cleaned by a cleaning tool.

As described above, the elastic sealing device 10 including the watertight holding section 20 and the packing section 40 is arranged in a predetermined position between the first frame body 60 and the second frame body 70 to integrally fix the first frame body 60 and the second frame body 70. Thereby, the first frame body 60 and the second frame body 70 in the engaging state due to the watertight holding section 20 of the elastic sealing device 10 can obtain the watertight holding state. At the same time, the first frame body 60 and the second frame body 70 can obtain the close contact state in which the packing main body 41 of the packing section 40 of the elastic sealing device 10 is arranged between the second engaging flat surface 73 and the first engaging flat surface 63 to thereby eliminate the gap.

In the endoscope after use, autoclave sterilization treatment is implemented as a sterilization treatment. During the sterilization treatment, the endoscope operation section configured as described above is exposed to a high-temperature and high-pressure atmosphere. Therefore, as shown in an open arrow Y4a in FIG. 4D, the first frame body 60 thermally expands in the longitudinal axis direction. On the other hand, as shown in an open arrow Y4b in FIG. 4D, the second frame body 70 thermally expands in the longitudinal axis direction. The first frame body 60 and the second frame body 70 thermally expand, and thereby respective positions of the first engaging flat surface 63 and the second engaging flat surface 73 are moved from a position shown in a solid line up to a position shown in a broken line to thereby further compress the packing main body 41.

At this time, the packing section 40 of the elastic sealing device 10 is brought into a state of being compressed to the size A and biting into the second ridge line 74b and is pressed and held between the first frame body 60 and the second frame body 70. Therefore, the packing main body 41 and the joining section 42 arranged in the third gap S3 are extended to the O-shaped ring shape section 21 side.

Then, the thin wall section 30 is pressed and deformed in a direction of the bottom of the circumferential groove 66 along with the extension to the O-shaped ring shape section 21 side. Therefore, the O-shaped ring shape section 21 keeps the watertight holding state without being extended.

As described above, the packing main body 41 compressed to a thickness in an elastic range assumed in advance is arranged between the first frame body 60 and the second frame body 70 thermally expanded during the autoclave sterilization treatment. Therefore, the first engaging flat surface 63 and the second engaging flat surface 73 can be certainly prevented from being directly contacted with each other. Further, breakage of the first frame body 60 and the second frame body 70 due to the autoclave sterilization treatment can be reduced.

Note that, according to the embodiment described above, the second engaging flat surface 73 of the second frame body 70 is set to a flat surface that intersects with the second longitudinal axis c70 and a vertical flat surface orthogonal to the second longitudinal axis c70. However, the second frame body 70 shown in FIG. 4E includes a second engaging flat surface 73A having an inclined flat surface that intersects with the longitudinal axis c70.

As described above, the first ridge line 74a abuts on the O-shaped ring shape section 21 projected from the engaging side outer peripheral surface 65, and then the second frame body 70 is further moved to the first engaging flat surface 63 while squeezing the O-shaped ring shape section 21. Then, the second engaging flat surface 73A that is an inclined surface abuts on the other surface 41b side of the packing main body 41 of the elastic sealing device 10 as shown in a broken line.

Then, the operator moves the second engaging flat surface 73A shown in a broken line in FIG. 4F to the first engaging flat surface 63. Then, while a contact surface between the second engaging flat surface 73A that is the inclined flat surface and the other surface 41b of the packing main body 41 is gradually widened, the operator presses the packing main body 41. As a result, the packing main body 41 is compressed and deformed so as to be made finer in the shape on the outer side along the second engaging flat surface 73A shown in a solid line. On the other hand, the packing main body 41 is extended and deformed along with the compression.

The packing main body 41 is brought into the state of biting into the second ridge line 74b as described above and is extended to the O-shaped ring shape section 21 side. Thereby, the thickness T of the part of the packing main body 41 and the part of the joining section 42 arranged in the third gap S3 is expanded to the size B. Further, the packing main body 41 and the joining section 42 are deformed so as to be attached closely to the second engaging inclined surface 74 of the second frame body 70 and further the packing section 40 of the elastic sealing device 10 is stably and securely pressed and held between the first frame body 60 and the second frame body 70.

Further, the close contact state of eliminating the gap is brought between the second engaging flat surface 73A and the other surface 41b of the packing main body 41 and between the one surface 41a of the packing main body 41 and the first engaging flat surface 63. Further, a surface on the outer side of the packing main body 41 arranged between the first frame body 60 and the second frame body 70 configures the seal member outer peripheral surface 10o.

According to the configuration, similarly to the above-described embodiment, the first frame body 60 and the second frame body 70 in the engaging state can be brought into the watertight holding state. At the same time, the packing main body 41 on the outer side is compressed and deformed so as to be made thin in the shape by the second engaging flat surface 73A that is the inclined flat surface. Thereby, by a load at the time of being charged in an autoclave and the like, the packing main body 41 can be prevented from protruding to the outer side of the first frame body 60 and the outer side of the second frame body 70. At the same time, breakage of the first frame body 60 and the second frame body 70 due to the autoclave sterilization treatment can be reduced.

Note that, according to the above-described embodiment, in the assembled state, the elastic sealing device 10 arranged between the first frame body 60 and the second frame body 70 is compressed so that a thickness of the joining section 42 of the packing section 40 becomes larger than a thickness of the packing main body 41. Further, the packing section 40 of the elastic sealing device 10 is stably pressed and held between the first frame body 60 and the second frame body 70. However, in the assembled state, the elastic sealing device 10 arranged between the first frame body 60 and the second frame body 70 may be compressed so that a compression rate of the joining section 42 becomes larger than a compression rate of the packing main body 41. Further, the packing section 40 of the elastic sealing device 10 may be stably pressed and held between the first frame body 60 and the second frame body 70.

As described above in the present embodiment, the first ridge line 74a of the second frame body 70 first abuts on the O-shaped ring shape section 21 projected from the engaging side outer peripheral surface 65 as shown in a chain double-dashed line in FIG. 4G. Subsequently, the second frame body 70 is further moved to the first engaging flat surface 63 while squeezing the O-shaped ring shape section 21. Then, the second engaging inclined surface 74 abuts on the other surface 42b side of the joining section 42 of the elastic sealing device 10 as shown in a broken line.

In a state in which the second engaging inclined surface 74 is arranged abutting on the other surface 42b, the second engaging flat surface 73 is moved to the first engaging flat surface 63. Then, the second engaging flat surface 73 does not abut on the other surface 41b of the packing main body 41 but the second engaging inclined surface 74 abuts on the other surface 42b of the joining section 42.

Here, the second engaging flat surface 73 is further caused to move to the first engaging flat surface 63. Then, the second engaging flat surface 73 abuts on the other surface 41b of the packing main body 41, and on the other hand, the joining section 42b is compressed and deformed by the second engaging inclined surface 74. Then, the first frame body 60 and the second frame body 70 are brought into a predetermined assembled state. At this time, the joining section 42b is compressed by a size b and is deformed to have a thickness B1 and the packing main body 41 is compressed by a size a so that the thickness is changed to A1. At this time, a relationship between the size A1 and a size B1 satisfies an expression A1>B1. In other words, the joining section 42b is compressed in comparison to the packing main body 41.

When the joining section 42b is compressed as described above, the joining section 42b is extended and deformed along with the compression. In other words, the joining section 42 is extended to the packing main body 41 side and the O-shaped ring shape section 21 side from the second ridge line 74b in which the second engaging flat surface 73 and the second engaging inclined surface 74 intersect with each other. Then, as the joining section 42 is extended to the packing main body 41 side, the packing main body 41 is expanded and deformed so as to be firmly attached closely to the second engaging flat surface 73.

As a result, the packing section 40 of the elastic sealing device 10 is brought into a state of biting into the second ridge line 74b. Further, the elastic sealing device 10 is stably pressed and held between the first frame body 60 and the second frame body 70.

As described above, the relationship between the size A1 and the size B1 satisfies the expression A1>B1. However, according to the present embodiment, a compression rate C1 of the packing main body 41 is equal to a/(a+A1) and a compression rate C2 of the joining section 42 is equal to b/(b+B1). In other words, an expression C1<C2 holds between the compression rate C1 of the packing main body 41 and the compression rate C2 of the joining section 42.

As described above, a relationship of the expression C1<C2 is set between the packing main body 41 and the joining section 42 configuring the packing section 40. Thereby, in a state in which the first frame body 60 and the second frame body 70 are assembled, the joining section 42 is firmly pinched by a high contact pressure between the first frame body 60 and the second frame body 70. Further, the joining section 42 is stably pressed and held between the first frame body 60 and the second frame body 70.

Note that the close contact state of eliminating the gap is brought between the second engaging flat surface 73 and the other surface 41b of the packing main body 41 and between the one surface 41a of the packing main body 41 and the first engaging flat surface 63. At this time, a surface on the outer side of the packing main body 41 arranged between the first frame body 60 and the second frame body 70 configures the seal member outer peripheral surface 10o similar to the above-described seal member outer peripheral surface 10o.

Note that, according to the embodiment described above, in the elastic sealing device 10, the operator sets a thickness of the thin wall section 30 coupling the watertight holding section 20 and the joining section 42 to t. Further, the operator sets thicknesses of the packing main body 41 and the joining section 42 configuring the packing section 40 to the same thickness T.

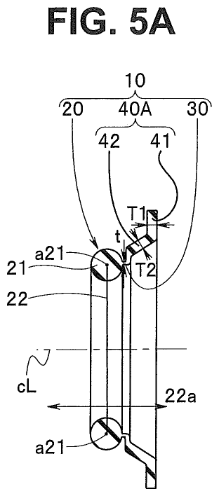

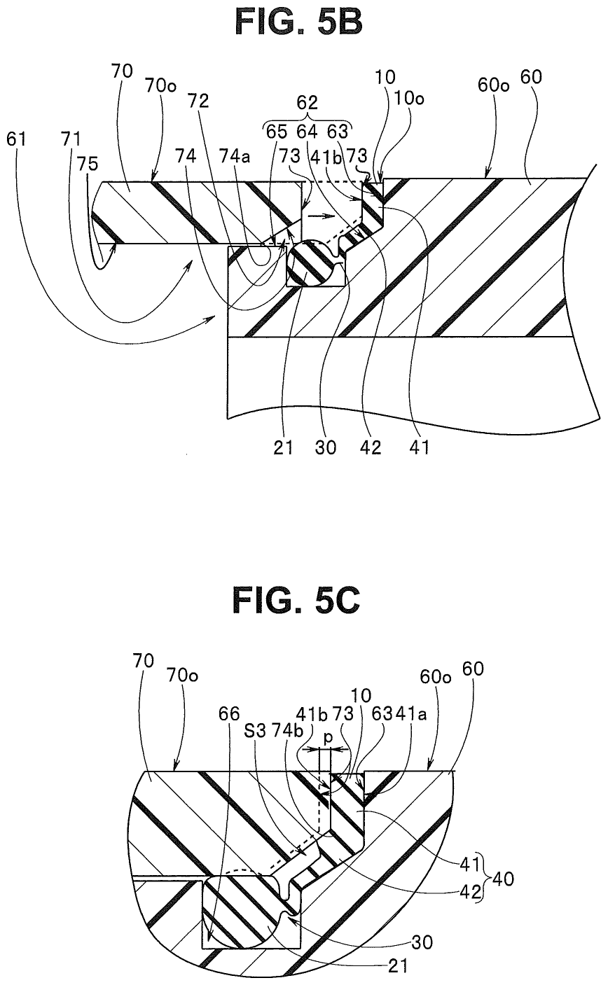

However, as shown in FIG. 5A, a packing section 40A of the elastic sealing device 10 may be configured. The packing section 40A includes the packing main body 41 a thickness of which is T1 and the joining section 42 a thickness of which is T2. Respective thicknesses are set so as to be smaller in the order corresponding to the packing main body 41, the joining section 42, and the thin wall section 30.

As shown in FIG. 5B, the elastic sealing device 10 having the packing section 40A is arranged on the first frame engaging surface 62 of the engaging convex section 61 by the operator as described above. Further, the engaging convex section 61 of the first frame body 60 is arranged in the engaging concave section 71 of the second frame body 70 by the operator. The operator causes the second engaging flat surface 73 to move to the first engaging flat surface 63 as shown in an arrow. Then, the first ridge line 74a abuts on the O-shaped ring shape section 21 projected from the engaging side outer peripheral surface 65.

Here, the operator moves the second engaging flat surface 73 to the first engaging flat surface 63 so that the O-shaped ring shape section 21 is squeezed and the second engaging flat surface 73 is moved as shown in a broken line. Further, the second engaging flat surface 73 abuts on the other surface 41b of the packing main body 41 of the elastic sealing device 10.

The O-shaped ring shape section 21 is squeezed and thereby can be attached closely to an inner face of the circumferential groove 66 and the engaging side inner peripheral surface 75. Further, the first frame body 60 and the second frame body 70 in the engaging state can be brought into the watertight holding state.

Here, as shown in FIG. 5C, the operator causes the second engaging flat surface 73 to slightly move to the first engaging flat surface 63 by p so as to be brought into a state shown in a solid line from a state shown in a broken line. Further, the operator causes the second engaging flat surface 73 to be pressed and arranged on the other surface 41b of the packing main body 41.

Then, the close contact state of eliminating the gap is brought between the second engaging flat surface 73 and the other surface 41b of the packing main body 41 and between the one surface 41a of the packing main body 41 and the first engaging flat surface 63.

When the close contact state is brought, the packing main body 41 is compressed, and on the other hand, is extended along with the compression. In other words, as shown in FIG. 5C, the packing main body 41 is extended to the O-shaped ring shape section 21 side from the second ridge line 74b in which the second engaging flat surface 73 and the second engaging inclined surface 74 intersect with each other.

Briefly, the part of the packing main body 41 and the part of the joining section 42 arranged in the third gap S3 are deformed so as to be attached closely to the second engaging inclined surface 74 of the second frame body 70. In other words, the elastic sealing device 10 is brought into a state in which the packing section 40 bites into the second ridge line 74b and is stably pressed and held between the first frame body 60 and the second frame body 70.

As described above, the elastic sealing device 10 that is set so that the thickness becomes smaller in the order corresponding to the packing main body 41, the joining section 42, and the thin wall section 30 is arranged between the first frame body 60 and the second frame body 70, and further the first frame body 60 and the second frame body 70 are brought into the assembled state. Also thereby, the elastic sealing device 10 is stably pressed and held between the first frame body 60 and the second frame body 70 to thereby obtain the action effect similar to the above-described action effect.

Note that the endoscope after use is exposed to a high-temperature and high-pressure atmosphere during the sterilization treatment. Then, the first frame body 60 and the second frame body 70 are thermally expanded and the packing main body 41 is compressed. At this time, the packing section 40 is brought into a state of biting into the second ridge line 74b and the elastic sealing device 10 is pressed and held between the first frame body 60 and the second frame body 70.

Therefore, the packing main body 41 and the joining section 42 arranged in the third gap S3 are extended to the O-shaped ring shape section 21 side. Accordingly, the packing main body 41 is slightly deformed to the outside and the seal member outer peripheral surface 10o is not projected to the outside. Further, the packing main body 41 is mainly extended to the O-shaped ring shape section 21 side while attached closely to the first engaging inclined surface 64 and the second engaging inclined surface 74 forming the third gap S3.

As described above, the packing main body 41 compressed to a thickness in an elastic range assumed in advance is arranged between the first frame body 60 and the second frame body 70 thermally expanded during the autoclave sterilization treatment. Therefore, the first engaging flat surface 63 and the second engaging flat surface 73 can be certainly prevented from being directly contacted with each other. Further, breakage of the first frame body 60 and the second frame body 70 due to the autoclave sterilization treatment can be reduced.

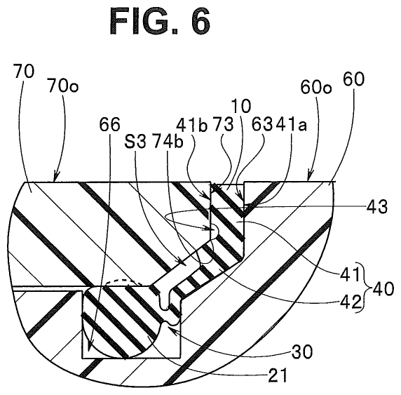

Note that, as shown in FIG. 6, a thinned concave section 43 may be provided in the vicinity of the joining section 42 of the packing main body 41 of the packing section 40 configuring the elastic sealing device 10a. According to the configuration, when the packing main body 41 is compressed by a thermal expansion of the first frame body 60 and the second frame body 70, the packing main body 41 is first extended and deformed so that a shape of the thinned concave section 43 is gradually thinned.

As a result, at the time of starting the compression, since the packing main body 41 is compressed by a small force, a deterioration of the packing main body 41 can be reduced.

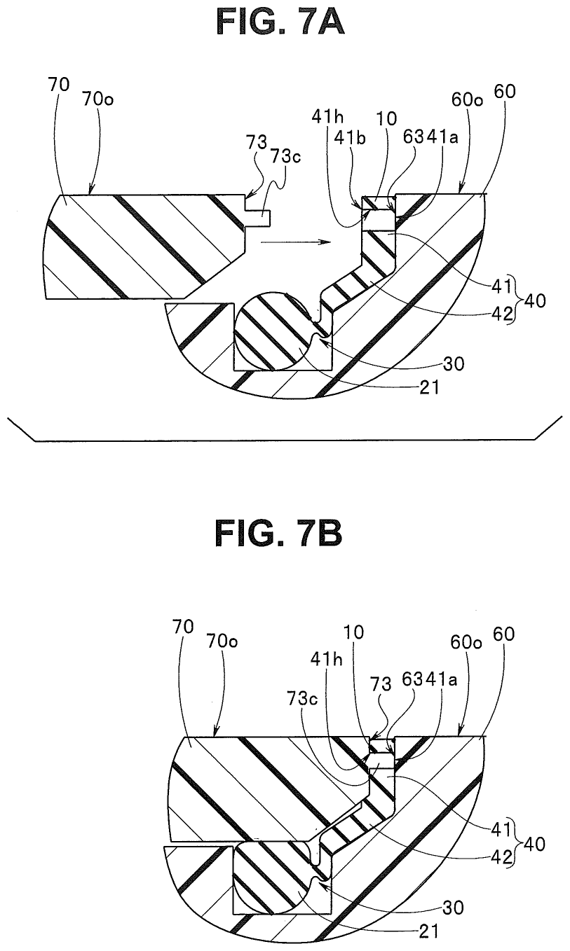

Note that, in addition to the first frame body 60 and the second frame body 70, the packing section 40 of the elastic sealing device 10 is configured as shown in FIGS. 7A, 8A, and 9A. Further, by a load at the time of being charged in an autoclave or the like, the packing main body 41 may be prevented from protruding to the outer side of the first frame body 60 and the outer side of the second frame body 70.

In FIG. 7A, a plurality of through-holes 41h are provided in a circumferential direction in predetermined positions of the packing main body 41. On the other hand, convex sections 73c that are arranged in the plurality of through-holes 41h, respectively, are provided on the second engaging flat surface 73 of the second frame body 70.

According to the configuration, as shown in FIG. 7B, in the assembled state between the first frame body 60 and the second frame body 70, the inner peripheral surfaces of the through-holes 41h are substantially arranged in close contact with the convex sections 73c, respectively. Further, the packing main body 41 is mechanically engaged with the second frame body 70, and thereby the packing main body 41 can be prevented from protruding to the outer side of the first frame body 60 and the outer side of the second frame body 70.

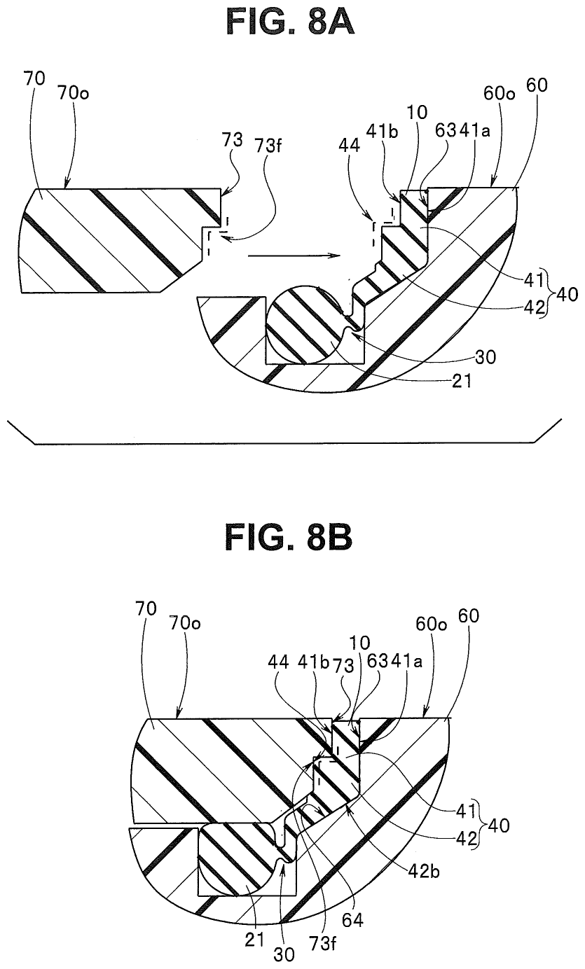

In FIG. 8A, in the packing section 40, a crank shape surface 44 is provided on the second frame body 70 side between the packing main body 41 and the joining section 42. Further, a crank shape restricting surface 73f that is arranged in contact with the crank shape surface 44 is provided on the second frame body 70.

According to the configuration, in the assembled state between the first frame body 60 and the second frame body 70 as shown in FIG. 8B, the crank shape restricting surface 73f presses the crank shape surface 44 and causes one side surface of the joining section 42 to be pressed and arranged on the first engaging inclined surface 64. In addition, the packing main body 41 is mechanically engaged with the second frame body 70, and thereby the packing main body 41 can be prevented from protruding to the outer side of the first frame body 60 and the outer side of the second frame body 70.

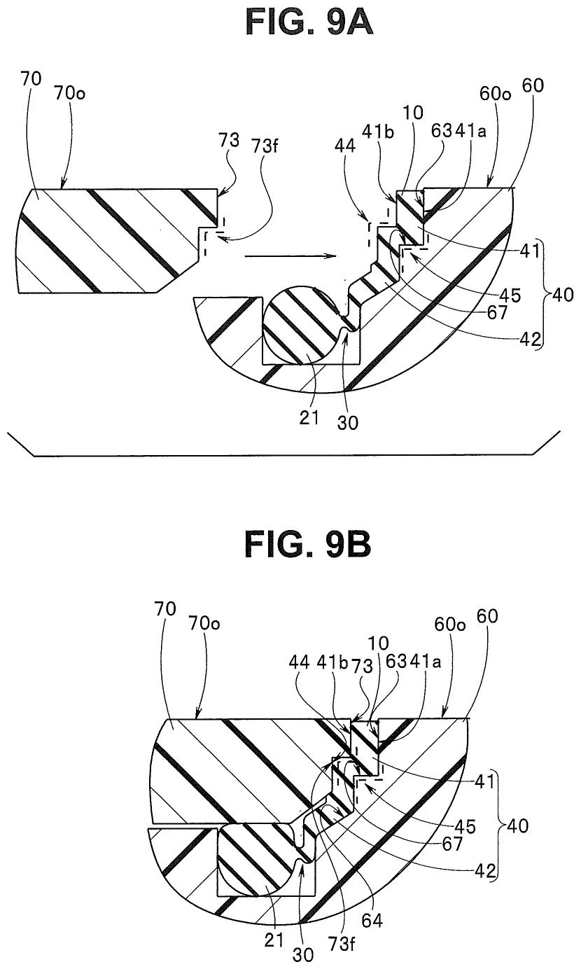

In FIG. 9A, in the packing section 40, a second crank shape surface 45 is provided on the first frame body 60 side in addition to the first crank shape surface 44 on the second frame body 70 side between the packing main body 41 and the joining section 42. Further, the crank shape restricting surface 73f that is arranged in contact with the first crank shape surface 44 is provided on the second frame body 70. Further, a fixing surface 67 for arranging the second crank shape surface 45 is provided on the first frame body 60.

According to the configuration, in the assembled state between the first frame body 60 and the second frame body 70, the crank shape restricting surface 73f presses the first crank shape surface 44 and causes the second crank shape surface 45 to be pressed and arranged on the crank shape fixing surface 67. In addition, the packing main body 41 is mechanically engaged with the second frame body 70, and thereby the packing main body 41 can be prevented from protruding to the outer side of the first frame body 60 and the outer side of the second frame body 70.

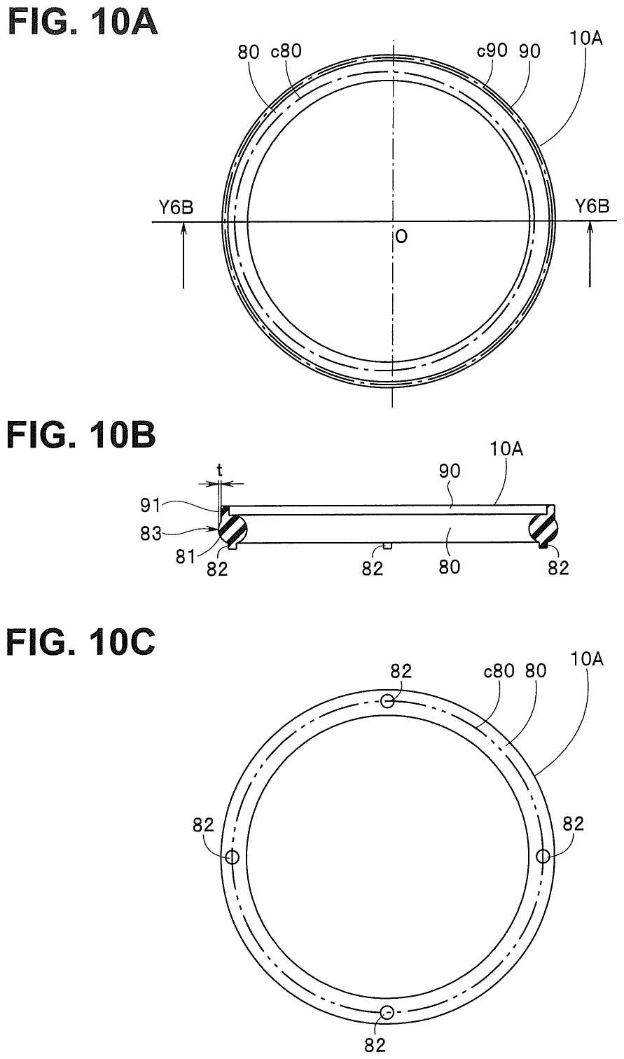

An elastic sealing device 10A will be described with reference to FIGS. 10A, 10B, and 10C.

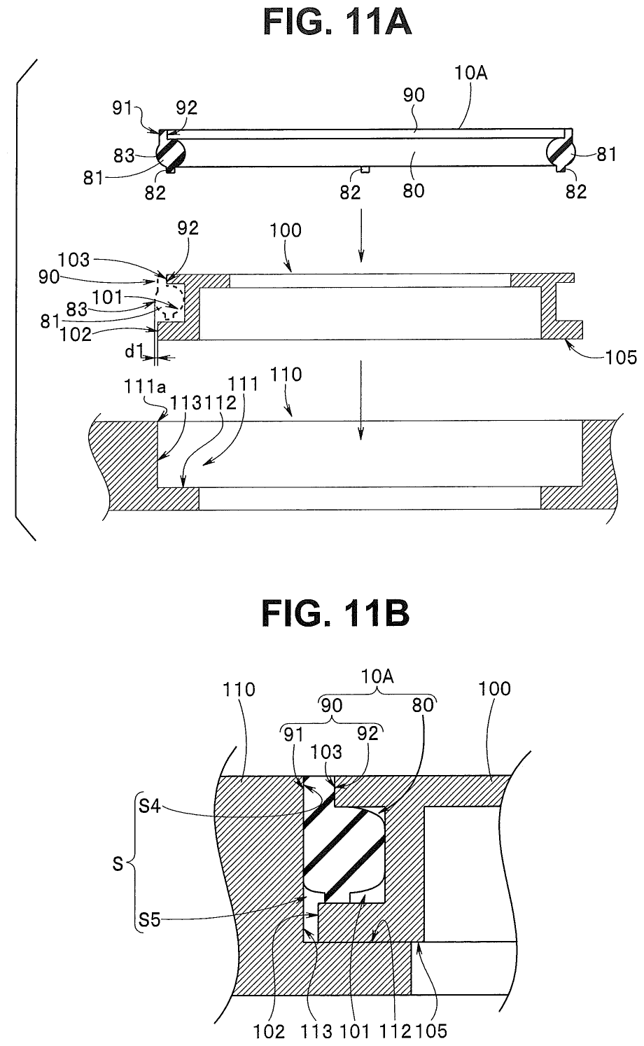

As shown in FIGS. 10A, 10B, and 10C, the elastic sealing device 10A mainly includes a watertight holding section 80 and a packing section 90 and is formed in a substantially ring shape. The elastic sealing device 10A is attached to a circumferential groove 101 of a frame body 100 shown in FIG. 11A.

As shown in FIG. 10B, the watertight holding section 80 includes an O-shaped ring shape section 81 a cross-sectional shape of which is a substantially round shape. The packing section 90 is projectingly provided on a front side that is one side of the O-shaped ring shape section 81. A front/back determination convex section 82 is provided on a back side that is the other side of the O-shaped ring shape section 81.

As shown in FIGS. 10B and 10C, the front/back determination section 82 is a convex section having a hemispherical shape, etc. and is provided in plurality in a circumferential direction, for example, at regular intervals. According to the present embodiment, four front/back determination sections 82 are provided.

According to the configuration, when the elastic sealing section 10A is grasped by fingers, a finger pulp is touched with the front/back determination section 82 and thereby a determination of the front side or the back side can be performed instantaneously. As a result, a direction of the elastic sealing section 10A is prevented from being misrecognized at the time of the attachment.

As shown in FIG. 10B, a cross-sectional shape of the packing section 90 is a rectangular shape and the packing section 90 is provided on an outer peripheral side of the O-shaped ring shape section 81, for example. An outside surface 91 of the packing section 90 is positioned on a central side of a predetermined size (refer to t in FIG. 10B) in consideration of an elastic deformation amount of the O-shaped ring shape section 81 from an outside surface 83 of the watertight holding section 80. Further, as shown in FIG. 10A, a central line c90 of the packing section 90 is shifted to the outside from a central line c80 of the watertight holding section 80.

Note that, in the figure, the packing section 90 is supposed to be provided on the outer peripheral side of the O-shaped ring shape section 81. However, the central line c90 of the packing section 90 may be caused to be shifted to the inner side from the central line c80 of the watertight holding section 80 and the packing section 90 may be provided on the inner peripheral side of the O-shaped ring shape section 81.

According to the configuration, when the elastic sealing device 10A is twisted and reversed, a position of the packing section 90 is shifted to the central line c80 of the watertight holding section 80. Thereby, a twisted state is not stably held and a reaction force to be returned to an original state is applied. As a result, the elastic sealing device 10A is not reversed and returned to the original state.

Note that even if the elastic sealing device 10A is left to be reversed by any possibility, it is visually recognized that the packing section 90 projecting from the watertight holding section 80 is in the twisted state and it is possible to instantaneously recognize a failure. Therefore, the elastic sealing device 10A can be prevented from being used in the twisted state.

The elastic sealing device 10A described above is attached to the circumferential groove 101 of the first frame body 100 shown in FIG. 11A. The first frame body 100 includes a first engaging side outer peripheral surface 102 including the circumferential groove 101 and a first engaging surface 103.

The O-shaped ring shape section 81 is arranged in the circumferential groove 101 as shown in a broken line and an inner surface 92 of the packing section 90 is arranged in the first engaging surface 103.

The circumferential groove 101 is formed in predetermined width and depth. The outer surface 83 of the O-shaped ring shape section 81 arranged in the circumferential groove 101 projects to the outside from the first engaging side outer peripheral surface 102 by a predetermined amount (refer to dl in FIG. 11A).

The first frame body 100 in which the elastic sealing device 10A is arranged is arranged in an engaging concave section 111 of a second frame body 110. A first frame body support surface 112 that is a bottom surface and a second engaging surface 113 are provided in the engaging concave section 111.

When the first frame body 100 having the elastic sealing device 10A is caused to be arranged in the engaging concave section 111 of the second frame body 110, one surface 105 of the first frame body 100 is caused to move to the first frame body support surface 112. At this time, a concave section ridge line 111a abuts on the O-shaped ring shape section 81 and then the O-shaped ring shape section 81 is squeezed by the second engaging surface 113. On the other hand, the packing section 90 is extended.

Then, as shown in FIG. 11B, the one surface 105 of the first frame body 100 reaches the first frame body support surface 112. Further, the packing section 90 is arranged in a first gap S4 in which the first engaging surface 103 and the second engaging surface 113 face with each other. Further, the O-shaped ring shape section 81 is arranged in a second gap S5 in which the first engaging side outer peripheral surface 102 and the second engaging surface 113 face with each other. At this time, the O-shaped ring shape section 81 of the elastic sealing device 10A is attached closely to an inner face of the circumferential groove 101 and the second engaging surface 113 to thereby hold the watertight holding state. On the other hand, the packing section 90 of the elastic sealing device 10 is arranged without gaps on the first engaging surface 103 and the second engaging surface 113 to be brought into a watertight state.

In other words, the first frame body 100 in which the elastic sealing device 10A having the watertight holding section 80 and the packing section 90 is provided is put in an engaged state with the second frame body 110. At this time, while surely securing the watertight state between the first frame body 100 and the second frame body 110, a surface of the elastic sealing device 10A, a surface of the first frame body 100, and a surface of the second frame body 110 can be configured as a continuous outer surface without steps. Alternatively, a range that a brushing section of a cleaning brush reaches is made concave to thereby attain an improvement in cleanability.

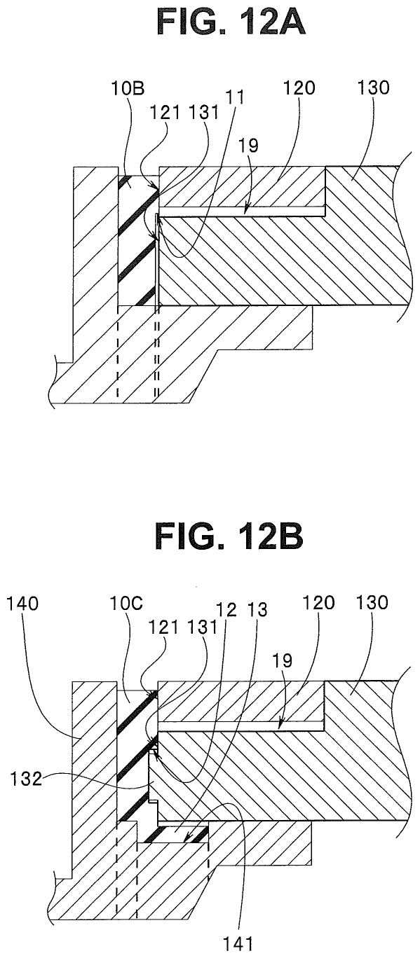

Note that as shown in FIG. 12A, for example, an elastic sealing device 10B may be arranged on a surface 121 of the first frame body 120 arranged most externally and a surface 131 of a second frame body 130 arranged on the inside.

In such a case, an escape section 11 is provided in a portion facing the surface 131 of the second frame body 130 of the elastic sealing device 10B.

As a result, an influence of the elastic sealing device 10B being compressed by a thermal expansion of the second frame body 130 or the like is not received. Further, the elastic sealing device 10B can be maintained in a preferable state to the surface 121 of the first frame body 120 arranged most externally. Note that a reference numeral 19 represents a screwing section and the first frame body 120 and the second frame body 130 are screw-fixed.

Further, it is necessary to regulate an arrangement position of an elastic sealing device 10C. In such a case, when a convex section 132 is provided on the surface 131 of the frame body 130, for example, as shown in FIG. 12B, a positioning concave section 12 capable of housing the convex section 132 is provided on the elastic sealing device 10C. Thereby, an arrangement state regulated by housing the convex section 132 in the concave section 12 is obtained.

Further, in a case in which one surface of the elastic sealing device 10C is arranged also in a third frame body 140, a shape of a surface arranged in the third frame body 140 of the elastic sealing device 10C is caused to coincide with a shape of an arrangement surface 141. In other words, an attachment section 13 or the like is provided in the elastic sealing device 10C in consideration of a working property apart from an object for obtaining the watertight state, the close contact state, or the like. Thereby, it is possible to attain an improvement in the working property.

The present invention is not limited to the embodiments described above, and various changes and modifications may be made without departing from the spirit or scope of the present invention.

What is claimed is:

1. A fixing structure of an elastic sealing device, comprising:
an elastic sealing device including a watertight holding section that is provided between adjacent frame bodies and holds watertightness and a packing section that is projectingly provided with respect to the watertight holding section and that is arranged in a gap between the adjacent frame bodies and forms at least a part of an outer surface;
a first frame body including a first frame outer peripheral surface that is exposed to an outside and is an outer surface and a first frame engaging surface in which the elastic sealing device is arranged; and
a second frame body including a second frame outer peripheral surface that is exposed to the outside and is an outer surface and a second frame engaging surface that is arranged facing the first frame engaging surface, and in which a gap for arranging the elastic sealing device is formed between the first frame engaging surface and the second frame engaging surface by being assembled into the first frame body.

2. The fixing structure of the elastic sealing device according to claim 1, wherein
the first frame engaging surface includes a first engaging surface in which the packing section is arranged and an engaging side outer peripheral surface in which the watertight holding section is arranged,
the second frame engaging surface includes a second engaging surface facing the first engaging surface and an engaging side inner peripheral surface facing the engaging side outer peripheral surface, and
in a state in which the first frame body and the second frame body are assembled,
the gap includes a first gap in which the first engaging surface and the second engaging surface face each other; and
a second gap in which the engaging side outer peripheral surface and the engaging side inner peripheral surface face each other.

3. The fixing structure of the elastic sealing device according to claim 1, wherein
the packing section includes:
a first portion that is a part of the outer surface; and
a second portion that is positioned between the first portion and the watertight holding section.

4. The fixing structure of the elastic sealing device according to claim 3, wherein
in the packing section, a thickness of the second portion is larger than a thickness of the first portion in a compressed state.

5. The fixing structure of the elastic sealing device according to claim 3, wherein
in the packing section, a compression rate of the second portion is larger than a compression rate of the first portion in the compressed state.

6. The fixing structure of the elastic sealing device according to claim 3, wherein
the first portion includes a first thickness and the second portion includes a second thickness, and
the first thickness is formed more thickly than the second thickness.

7. The fixing structure of the elastic sealing device according to claim 3, wherein
the second portion and the watertight holding section are coupled by a thin wall section that is formed more thinly than the first portion and the second portion.

8. The fixing structure of the elastic sealing device according to claim 3, wherein
a thinned concave section is provided in a portion coupling the first portion and the second portion of the packing section.

9. An elastic sealing device comprising:
a watertight holding section that is provided between adjacent frame bodies and holds watertightness; and
a packing section that is projectingly provided with respect to the watertight holding section and that is arranged in a gap between the adjacent frame bodies and forms at least a part of an outer surface, wherein
the watertight holding section includes, in a circumferential direction, a plurality of convex sections that project in a direction opposite to the packing section from the watertight holding section.

10. An elastic sealing device comprising:
a watertight holding section that is provided between adjacent frame bodies and holds watertightness; and
a packing section that is projectingly provided with respect to the watertight holding section and that is arranged in a gap between the adjacent frame bodies and forms at least a part of an outer surface, wherein
the packing section is a circumferential projection that projects from the watertight holding section in one direction and is provided along an outer peripheral surface or an inner peripheral surface of the watertight holding section, and
a central line of the circumferential projection and a central line of the watertight holding section do not match each other.

11. An endoscope in which the fixing structure of the elastic sealing device according to claim 1 is provided in at least an endoscope operation section or an endoscope connector.

12. An endoscope in which the elastic sealing device according to claim 9 is provided in at least an endoscope operation section or an endoscope connector.

13. An endoscope in which the elastic sealing device according to claim 10 is provided in at least an endoscope operation section or an endoscope connector.
